(19) United States
(12) Patent Application Publication
(10) Pub. No.: US 2025/0019327 A1
(43) Pub. Date: Jan. 16, 2025

(54) METHOD FOR PRODUCING TERTIARY BUTYL ALCOHOL

(71) Applicant: MITSUBISHI CHEMICAL CORPORATION, Tokyo (JP)

(72) Inventors: Shunsuke HASHIMOTO, Tokyo (JP); Takafumi TOYODA, Tokyo (JP); Yugo MIZOKOSHI, Tokyo (JP); Atsushi NAGIRA, Tokyo (JP)

(73) Assignee: MITSUBISHI CHEMICAL CORPORATION, Tokyo (JP)

(21) Appl. No.: 18/898,518

(22) Filed: Sep. 26, 2024

Related U.S. Application Data
(63) Continuation of application No. PCT/JP2023/011523, filed on Mar. 23, 2023.

(30) Foreign Application Priority Data
Mar. 28, 2022 (JP) 2022-052369

(51) Int. Cl. C07C 29/04; C07C 29/80

(52) U.S. Cl. CPC C07C 29/04; C07C 29/80

(57) ABSTRACT

A method for producing tert-butyl alcohol, including (i) a step of feeding a raw material liquid including isobutylene and water, to a reactor having a cation exchange resin, and (ii) a step of producing a reaction product including tert-butyl alcohol, by hydration reaction of isobutylene in the reactor, in which, in step (ii), the average superficial linear velocity of the raw material liquid, as calculated in the following expression (I), is 5 m/hour or more,

Average linear velocity of raw material liquid (m/hour)=Volume flow rate of raw material liquid (m³/hour)/Cross-sectional area of reactor (m²) (I), and

the volume of an aqueous phase (Vw) in a fluid (fluid A) to the total volume of the fluid A in the reactor is 4 to 12% by volume.

### CROSS-REFERENCE TO RELATED APPLICATION

This is a continuation of International Application PCT/JP2023/011523, filed on Mar. 23, 2023, and designated the U.S., and claims priority from Japanese Patent Application 2022-052369 which was filed on Mar. 28, 2022, the entire contents of which are incorporated herein by reference.

### TECHNICAL FIELD

The present invention relates to a method for producing tert-butyl alcohol (hereinafter, also referred to as “TBA”.).

### BACKGROUND ART

Tert-butyl alcohol is used for raw materials of methyl methacrylate production according to gas-phase catalytic oxidation methods.

As methods for producing tert-butyl alcohol, methods including hydration reaction of isobutylene (2-methylpropene) and water by use of catalysts are known. Such reaction generally corresponds to reaction in heterogeneous liquid phases of isobutylene and water phase-separated, because isobutylene and water as raw materials are less soluble in each other.

As methods for producing tert-butyl alcohol in heterogeneous liquid phases, for example, Patent Document 1 and Patent Document 2 each describe a method including contacting isobutylene and water on a strongly acidic cation exchange resin catalyst particle surface by a predetermined method.

However, the method in a heterogeneous liquid phase, as described in each of Patent Document 1 and Patent Document 2, causes a low reaction rate, a need for a large reactor for securement of the amount of production, and an increase in facility cost. Therefore, there is a demand for a procedure capable of realizing a higher reaction rate from an industrial viewpoint.

On the other hand, Patent Document 3 describes a method including reaction in a homogeneous liquid phase by use of an aqueous solution of an aliphatic carboxylic acid having 1 to 6 carbon atoms. However, this method requires a step of separating carboxylic acid ester of tert-butyl alcohol from a reaction liquid in order to obtain tert-butyl alcohol, and causes an increase in facility cost.

Patent Document 4 describes a method in which reaction is performed in a homogeneous liquid phase and a step of separating tert-butyl alcohol is included in midstream. However, this method includes adding an aqueous tert-butyl alcohol solution to a reactor to provide a homogeneous liquid phase, and thus the conversion rate is restricted due to equilibrium.

### RELATED ART DOCUMENT

### Patent Document

Patent Document 1: JP-A S54-030104
Patent Document 2: JP-A S54-30105
Patent Document 3: JP-B S60-051451
Patent Document 4: JP-A S60-233024

### SUMMARY OF THE INVENTION

### Problems to be Solved by the Invention

The present invention has been made in order to solve the above problems, and an object thereof is to provide a method for producing tert-butyl alcohol at a high conversion rate of isobutylene with suppressed facility cost and production cost.

### Means for Solving the Problems

The present inventors have made intensive studies in order to achieve the above object. As a result, the inventors have surprisingly found that a sufficient reaction rate is obtained by setting the linear velocity of a raw material liquid and the volume of an aqueous phase in a fluid in hydration reaction within specified ranges also in reaction in a heterogeneous liquid phase in which an isobutylene phase and an aqueous phase are separated, and thus have completed the present invention.

Specifically, the present invention includes the followings.

[1]: A method for producing tert-butyl alcohol, including
(i) a step of feeding a raw material liquid including isobutylene and water, to a reactor having a cation exchange resin, and
(ii) a step of producing a reaction product including tert-butyl alcohol, by hydration reaction of isobutylene in the reactor, wherein, in step (ii),
the average superficial linear velocity of the raw material liquid, as calculated in the following expression (I), is 5 m/hour or more,

Average linear velocity of raw material liquid (m/hour)=Volume flow rate of raw material liquid (m³/hour)/Cross-sectional area of reactor (m²) (I), and
the volume of an aqueous phase (Vw) in a fluid (fluid A) to the total volume of the fluid A in the reactor is 4 to 12% by volume.

[2]: The method for producing tert-butyl alcohol according to [1], wherein the average superficial linear velocity of the raw material liquid is 5 to 29 m/hour in step (ii).

[3]: The method for producing tert-butyl alcohol according to [1] or [2], wherein the average superficial linear velocity of the raw material liquid is 7 to 13 m/hour in step (ii).

[4]: The method for producing tert-butyl alcohol according to any of [1] to [3], wherein the Vw is 5 to 10% by volume in step (ii).

[5]: The method for producing tert-butyl alcohol according to any of [1] to [4], wherein the outlet temperature of the reactor is 75° C. or less in step (ii).

[6]: The method for producing tert-butyl alcohol according to any of [1] to [5], further including (iii) a step of separating unreacted isobutylene from the reaction product including tert-butyl alcohol, produced in step (ii), to produce tert-butyl alcohol.

[7]: The method for producing tert-butyl alcohol according to [6], wherein, when the amount of tert-butyl alcohol fed per unit time to the reactor in step (i) is defined as Mi (mol/hour) and the amount of tert-butyl alcohol produced per unit time in step (iii) is defined as Miii (mol/hour), Mi/Miii is 1 to 7.

[8]: The method for producing tert-butyl alcohol according to [7], wherein the Mi/Miii is 1 to 5.

### Effects of the Invention

According to the present invention, there can be provided a method for producing tert-butyl alcohol at a high conversion rate isobutylene with suppressed facility cost and production cost.

### BRIEF DESCRIPTION OF THE DRAWINGS

FIG. 1 illustrates one example of a reaction apparatus for carrying out the present invention.

FIG. 2 is a three-phase diagram representing the proportion of each component in a case where the total of hydrocarbon, water and TBA in a fluid A is 100% by mol. A shaded part represents a region in which the volume of an aqueous phase (Vw) in the fluid A based on the total volume of the fluid A in the fluid A is 4 to 12% by volume.

FIG. 3 is a three-phase diagram representing the proportion of each component in a case where the total of hydrocarbon, water and TBA in a fluid A is 100% by mol. A shaded part represents a region in which the volume of an aqueous phase (Vw) in the fluid A based on the total volume of the fluid A in the fluid A is 5 to 10% by volume.

### MODE FOR CARRYING OUT THE INVENTION

Hereinafter, embodiments according to the present invention are described, but the present invention is not limited to the following. Herein, a numerical value range expressed with “to” means a range including numerical values described before and after “to” respectively as the lower limit value and the upper limit value, and “A to B” means A or more and B or less.

A method for producing tert-butyl alcohol according to the present embodiment includes the following steps (i) and (ii).
(i) A step of feeding a raw material liquid including isobutylene and water, to a reactor having a cation exchange resin.
(ii) A step of producing a reaction product including tert-butyl alcohol, by hydration reaction of isobutylene in the reactor.

In the method for producing tert-butyl alcohol according to the present embodiment, in step (ii), the average superficial linear velocity of the raw material liquid, as calculated in the following expression (I), is 5 m/hour or more.

$$Average linear velocity of raw material liquid ( m / hour ) = Volume flow rate of raw material liquid ( {m}^{3} / hour ) \text{} / Cross - sectional area of reactor ( {m}^{2} ) \qquad ( I )$$

The volume of an aqueous phase (Vw) in a fluid (fluid A) to the total volume of the fluid A in the reactor is 4 to 12% by volume.

Such a method can be used to produce tert-butyl alcohol at a high conversion rate of isobutylene with suppressed facility cost and production cost.

The method for producing tert-butyl alcohol according to the present embodiment preferably further includes the following step (iii).
(iii) A step of separating unreacted isobutylene from the reaction product including tert-butyl alcohol, produced in step (ii), to produce tert-butyl alcohol.

Hereinafter, each step is described in detail.

[Step (i)]

In step (i), a raw material liquid including isobutylene and water is fed to a reactor having a cation exchange resin.

### <Reactor>

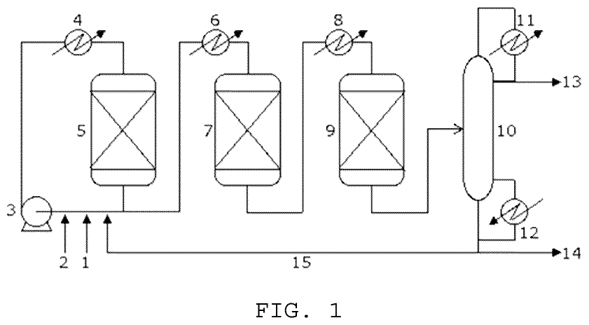

The reactor used in the present embodiment can be any of a stirring vessel-type reactor, a fixed bed-type reactor, a tower-type reactor, and the like. The reaction system may be any of a batch system, a semi-batch system, and a continuous flow system. The reactor may be provided in series or in parallel, and as illustrated in FIG. 1, a plurality of such reactors is preferably provided in series and 2 to 5 such reactors are more preferably provided in series.

In a case where a plurality of such reactors is present, the effects of the present invention can be obtained as long as the average linear velocity of the raw material liquid, and the Vw satisfy defined conditions in at least one of such reactors. Such a reactor, to which the raw material liquid is to be first fed, namely, a first reactor 5 illustrated in FIG. 1, preferably satisfies the above conditions from the viewpoint of the conversion rate of isobutylene. Hereinafter, in a case where a plurality of such reactors is present, the “reactor” herein means such a reactor, to which the raw material liquid is to be fed first, unless particularly noted.

The cation exchange resin is preferably a strongly acidic cation exchange resin. Examples include Lewatit (trade name) manufactured by Bayer AG and Amberlyst (trade name) manufactured by Du Pont.

The location of the cation exchange resin in the reactor, the proportion of the cation exchange resin in the reactor, and the like are not particularly limited, and any form commonly used can be applied.

In a reaction apparatus of FIG. 1, isobutylene and water as raw materials are fed respectively through an isobutylene feed port 1 and a water feed port 2, and the raw materials are transferred to a first reactor 5 by a transfer pump 3. The raw materials, when transferred to the first reactor 5, are cooled to a predetermined temperature by a raw material cooler 4.

A reaction product of the first reactor 5 not only is transferred to a second reactor 7, but also is partially fed again as a raw material. The reaction product of the first reactor 5, when transferred to the second reactor 7, is cooled to a predetermined temperature by a raw material cooler 6.

A reaction product of the second reactor 7 is cooled by a raw material cooler 8, and transferred to a third reactor 9.

A reaction product of the third reactor 9 is transferred to a TBA separation tower 10. In the TBA separation tower 10, unreacted isobutylene in the reaction product of the third reactor 9 is cooled by a condenser 11, and discharged through an unreacted isobutylene discharge port 13. In addition, TBA in the reaction product of the third reactor 9 not only is heated by a reboiler 12 and discharged through a TBA discharge port 14, but also is partially fed again as a raw material through a TBA circulation line 15 to the first reactor 5.

### <Raw Material Liquid>

In the present embodiment, the raw material liquid includes isobutylene and water. The concentration of isobutylene in the raw material liquid is preferably 4 to 35% by mol. When the concentration of isobutylene is 4% by mol or more, the reaction rate of hydration reaction is enhanced in step (ii) described below. When the concentration of isobutylene is 35% by mol or less, isobutylene is inexpensively obtained, and thus the production cost is suppressed. The lower limit of the concentration of isobutylene is more preferably 8% by mol or more and the upper limit thereof is more preferably 18% by mol or less.

The water is not particularly limited, and is preferably deionized water, distilled water, or the like, more preferably deionized water. Impurities in the water can cause deactivation of a catalyst and have an adverse effect on the product quality, and are preferably removed as much as possible.

The raw material liquid may include tert-butyl alcohol. Here, when the amount of tert-butyl alcohol fed per unit time to the reactor in step (i) is defined as Mi (mol/hour) and the amount of tert-butyl alcohol produced per unit time in step (iii) described below is defined as Miii (mol/hour), the Mi/Miii is preferably 1 to 7. Thus, the conversion rate of isobutylene is enhanced in step (ii) described below, and therefore the production cost can be suppressed in step (iii) described below. The upper limit of the Mi/Miii is more preferably 5 or less, further preferably 4.5 or less, particularly preferably 4 or less. Here, tert-butyl alcohol included in the raw material liquid may include recycled TBA with tert-butyl alcohol produced in step (iii) described below.

The Mi/Miii can be adjusted by, for example, modifying the feeding rate of the raw material liquid fed to the reactor, and the concentration of tert-butyl alcohol in the raw material liquid.

The raw material liquid may also include hydrocarbon or the like, in addition to the above. The hydrocarbon is preferably one or more selected from hydrocarbons each having 4 carbon atoms, other than isobutylene, such as butenes (1-butene and/or 2-butene) and butanes (n-butane, isobutane, and/or the like). Such isobutylene-containing hydrocarbon can be obtained as a by-product in formation of ethylene by pyrolysis of naphtha in the presence of water vapor, a by-product in catalytic contact cracking of heavy oil, or such a by-product from which butadiene is removed.

The raw material liquid is fed so that the average superficial linear velocity calculated by the expression (I) is 5 m/hour or more in step (ii) described below. Thus, in a case where the Vw is within a prescribed range in step (ii), the reaction rate of hydration reaction is enhanced. The reason for this is considered as follows.

In a case where the Vw is within a prescribed range in step (ii) described below, an aqueous phase is partially dispersed in an isobutylene phase and then formed into water droplets. When the raw material liquid is here fed at a defined rate, the water droplet diameter is decreased and the number of water droplets is increased. Thus, the area of a contact interface between an isobutylene phase and an aqueous phase in the fluid A is increased, and interphase mass transfer is promoted. The lower limit of the average linear velocity of the raw material liquid is preferably 7 m/hour or more, more preferably 8 m/hour or more. The upper limit thereof is preferably 29 m/hour, more preferably 15 m/hour, further preferably 14 m/hour, particularly preferably 13 m/hour, most preferably 12 m/hour.

[Step (ii)]

In step (ii), a reaction product including tert-butyl alcohol is produced by hydration reaction of isobutylene in the reactor.

### <Fluid in Reactor in Hydration Reaction>

The average superficial linear velocity of the raw material liquid, as calculated in the following expression (I), is 5 m/hour or more in step (ii).

$$Average linear velocity of raw material liquid ( m / hour ) = Volume flow rate of raw material liquid ( {m}^{3} / hour ) \text{} / Cross - sectional area of reactor ( {m}^{2} ) \qquad ( I )$$

The volume flow rate of the raw material liquid, here used, is the volume flow rate of the raw material liquid fed to the reactor in step (i). The cross-sectional area of the reactor is the cross-sectional area using an inserted object part in the reactor. The cross-sectional area of the reactor, if not homogeneous, is the cross-sectional area of a site having the largest cross-sectional area in the reactor.

The average superficial linear velocity of the raw material liquid can be adjusted by, for example, modifying the volume flow rate of the raw material liquid fed to the reactor in step (i).

The volume of the aqueous phase (Vw) in the fluid (fluid A) to the total volume of the fluid A in the reactor is 4 to 12% by volume in step (ii). The aqueous phase means a phase including 90% by mol or more of water. The fluid A which has the aqueous phase means that the reaction in the reactor is reaction in a heterogeneous liquid phase in which the aqueous phase and the isobutylene phase are separated.

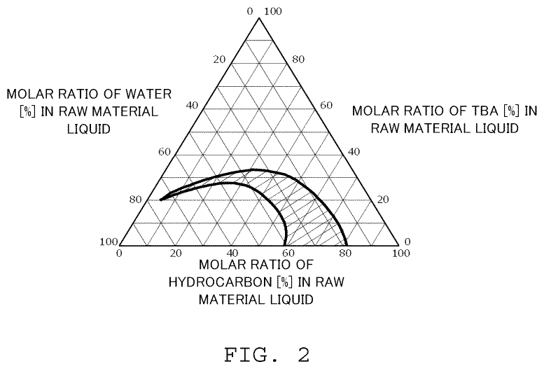

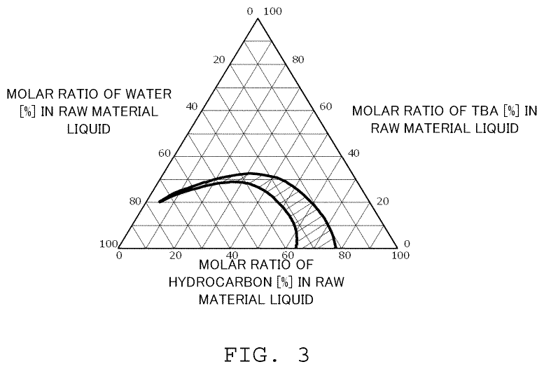

When the Vw is within a prescribed range, the reaction rate of isobutylene hydration reaction is enhanced. The reason for this is considered because the aqueous phase is partially dispersed in the isobutylene phase and present in the form of water droplets and thus interphase mass transfer with the isobutylene phase is promoted. The lower limit of the Vw is preferably 4% by volume or more, more preferably 5% by volume or more. The upper limit thereof is preferably 10% by volume or less, more preferably 9% by volume or less. The Vw can be adjusted by, for example, the concentration of tert-butyl alcohol included in the raw material liquid. The Vw can be 4 to 12% by volume by feeding the raw material liquid including tert-butyl alcohol so that the composition of the fluid A is within the range represented by the shaded section in FIG. 2. Similarly, the Vw can be 5 to 10% by volume by feeding the raw material liquid including tert-butyl alcohol so that the composition of the fluid A is within the range represented by the shaded section in FIG. 3.

It can be confirmed with FIG. 2 that the Vw is 4 to 12% by volume. The Vw can be determined to be 4 to 12% by volume from the composition of hydrocarbon, water and TBA of the raw material liquid including tert-butyl alcohol, if the composition of the fluid A is within the range represented by the shaded section in FIG. 2. Similarly, the Vw can be determined to be 5 to 10% by volume if the composition of the fluid A is within the range represented by the shaded section in FIG. 3.

The Vw can also be determined by the following method.

The density D of the fluid A (mol/m³), the density Dw of the aqueous phase (mol/m³) in the fluid A, and the density Do of the isobutylene phase (mol/m³) in the fluid A are calculated from the composition, the temperature and the pressure of the fluid A, with a process simulator. The process simulator here used can be, for example, Aspen Plus manufactured by Aspen Technology Inc. Next, the resulting values of D, Dw and Do can be used to solve the system of equations of the following expressions (II) and (III), thereby calculating the volume Vw of the aqueous phase (% by volume) in the fluid A based on the total volume of the fluid A, and the volume Vi of the isobutylene phase (% by volume) in the fluid A based on the total volume of the fluid A.

$$D = Dw \times Vw / 100 + Do \times Vo / 100 \qquad ( II )$$

$$Vw + Vi = 100 \qquad ( III )$$

### <Other Hydration Reaction Conditions>

The outlet temperature of the reactor in hydration reaction is preferably 75° C. or less. Thus, the amount of a by-product generated in hydration reaction can be suppressed. The reason for this is considered because a reaction temperature of 75° C. or less can allow a relative reaction rate of side reaction such as dimerization reaction of isobutylene to the reaction rate of hydration of isobutylene to be kept low. The lower limit of the outlet temperature of the reactor is more preferably 40° C. or more and the upper limit thereof is more preferably 65° C. or less.

The pressure in the reactor in hydration reaction is preferably 0.2 to 2.0 MPa (G). Herein, (G) represents the gauge pressure. Thus, isobutylene as a raw material is sufficiently liquefied. The lower limit of the pressure in the reactor is more preferably 0.4 MPa (G) and the upper limit thereof is more preferably 1.6 MPa (G). In order to keep the pressure in the reactor in hydration reaction, an inert gas not involving in hydration reaction may be introduced into the reactor. Examples of the inert gas include nitrogen and argon.

[Step (iii)]

In step (iii), unreacted isobutylene is separated from the reaction product including tert-butyl alcohol, produced in step (ii), to produce tert-butyl alcohol. If the conversion rate of isobutylene is high in step (ii), the production cost in step (iii) is suppressed.

A separation apparatus such as a distillation apparatus, an extraction apparatus, or a membrane separation apparatus can be used for separation of unreacted isobutylene, and a distillation apparatus is preferably used. The distillation apparatus here used is preferably a distillation tower or a flash drum. The distillation tower here used is a plate tower or a packed tower, preferably the plate tower.

In a case where the distillation tower is used as the separation apparatus, the temperature and the pressure are usually adopted at which unreacted isobutylene is gasified and tert-butyl alcohol is liquefied. The overhead temperature is preferably 20 to 70° C., and the bottom temperature is preferably 100 to 160° C. The overhead pressure is preferably 0.25 to 0.9 MPa (G).

As described above, tert-butyl alcohol can be produced at a high conversion rate of isobutylene with suppressed facility cost and production cost.

### EXAMPLES

Hereinafter, the present invention is described in detail with reference to Examples and Comparative Examples, but the present invention is not limited to these Examples. Herein, “part(s)” in Examples and Comparative Examples means part(s) by mass. A reaction apparatus illustrated in FIG. 1 was used. Each reactor was an insulation-type fixed bed-type reactor.

### (Average Linear Velocity of Raw Material Liquid)

The average superficial linear velocity of the raw material liquid was determined from the volume flow rate of the raw material liquid fed to the reactor, by the following expression (I).

$$Average linear velocity of raw material liquid ( m / hour ) = Volume flow rate of raw material liquid ( {m}^{3} / hour ) \text{} / Cross - sectional area of reactor ( {m}^{2} ) \qquad ( I )$$

The cross-sectional area of the reactor was here the cross-sectional area of the reactor from which an inserted object was removed.

### (Vw)

The Vw was determined by the following procedure.

The density D of the fluid A (mol/m³), the density Dw of the aqueous phase (mol/m³) in the fluid A, and the density Do of the isobutylene phase (mol/m³) in the fluid A were calculated from the composition, the temperature and the pressure of the fluid A, with Aspen Plus manufactured by Aspen Technology Inc. The outlet temperature and pressure of the first reactor 5 were used as the temperature and pressure of the fluid A. Next, the resulting values of D, Dw and Do were used to solve the system of equations of the following expressions (II) and (III), thereby calculating the volume Vw of the aqueous phase (% by volume) in the fluid A based on the total volume of the fluid A, and the volume Vi of the isobutylene phase (% by volume) in the fluid A based on the total volume of the fluid A.

$$D = Dw \times Vw / 100 + Do \times Vo / 100 \qquad ( II )$$

$$Vw + Vi = 100 \qquad ( III )$$

### (Amount of Reboiler Heating Steam)

The amount of reboiler heating steam was determined by measuring the amount of steam fed to the reboiler 12 with a flow meter.

### (Calculation of Conversion Rate of Isobutylene)

Each product in Examples and Comparative Examples was analyzed with gas chromatography (GC-2014 manufactured by Shimadzu Corporation, column: ULBON HR-20M 50 m, diameter: 0.32 mm, film thickness: 0.25 μm). The conversion rate of isobutylene was determined from the results of gas chromatography, by the following expression.

Conversion rate of isobutylene (%)=M2/M1×100

In the expression, M1 represents the number of moles of isobutylene fed per unit time, and M2 represents the number of moles of isobutylene reacted per unit time.

### Example 1

The reaction apparatus illustrated in FIG. 1 was used, and was filled with Amberlyst 15 manufactured by Du Pont, as a cation exchange resin. Here, the first reactor 5, the second reactor 7 and the third reactor 9 here used were each a fixed bed-type reactor, and the amount of the cation exchange resin for filling was 100 parts for the first reactor 5, and was 50 parts for each of the second reactor 7 and the third reactor 9.

A raw material liquid having a concentration of isobutylene of 12.0% by mol, a concentration of water of 33.6% by mol, and a concentration of TBA of 29.8% by mol was fed so that the average linear velocity in the first reactor 5 was a value shown in Table 1. The raw material liquid included recycled TBA, and the Mi/Miii was 4.15.

The reactor inlet temperature of the first reactor 5 was adjusted to 52.2° C., and hydration reaction of isobutylene was performed in conditions of a reactor outlet temperature of 65.0° C., a pressure of 0.876 MPa (G), and an average residence time of 0.634 hours. Next, 30.3% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 53.8° C., a pressure of 0.804 MPa (G), and an average residence time of 1.05 hours. Next, the total amount of the reaction product obtained in the second reactor 7 was fed to the third reactor 9. The reactor inlet temperature of the third reactor 9 was adjusted to 53.1° C., and hydration reaction of isobutylene was performed in conditions of a reactor outlet temperature of 56.3° C., a pressure of 0.800 MPa (G), and an average residence time of 1.04 hours. The Vw in the first reactor 5, and the total conversion rate of isobutylene in all the reactors are shown in Table 1.

The reaction product including TBA, obtained in the third reactor 9, was fed to the TBA separation tower 10, unreacted isobutylene was separated by distillation, and thus an aqueous TBA solution having a concentration of TBA of 61.1% by mol was obtained. The amount of reboiler heating steam required for production of 1000 parts of the aqueous TBA solution is shown in Table 1.

### Example 2

The reaction apparatus, the cation exchange resin and the amount thereof for filling were those as in Example 1.

A raw material liquid having a concentration of isobutylene of 11.7% by mol, a concentration of water of 31.4% by mol, and a concentration of TBA of 30.0% by mol was fed so that the average linear velocity in the first reactor 5 was a value shown in Table 1. The raw material liquid included recycled TBA, and the Mi/Miii was 4.34.

The reactor inlet temperature of the first reactor 5 was adjusted to 52.7° C., and hydration reaction of isobutylene was performed in conditions of a reactor outlet temperature of 64.9° C., a pressure of 0.875 MPa (G), and an average residence time of 0.618 hours. Next, 30.0% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 53.3° C., a pressure of 0.805 MPa (G), and an average residence time of 1.04 hours. Next, the total amount of the reaction product obtained in the second reactor 7 was fed to the third reactor 9. The reactor inlet temperature of the third reactor 9 was adjusted to 52.9° C., and hydration reaction of isobutylene was performed in conditions of a reactor outlet temperature of 55.5° C., a pressure of 0.800 MPa (G), and an average residence time of 1.03 hours. The Vw in the first reactor 5, and the total conversion rate of isobutylene in all the reactors are shown in Table 1.

The reaction product including TBA, obtained in the third reactor 9, was fed to the TBA separation tower 10, unreacted isobutylene was separated by distillation, and thus an aqueous TBA solution having a concentration of TBA of 63.9% by mol was obtained. The amount of reboiler heating steam required for production of 1000 parts of the aqueous TBA solution is shown in Table 1.

### Example 3

The reaction apparatus, the cation exchange resin and the amount thereof for filling were those as in Example 1.

A raw material liquid having a concentration of isobutylene of 16.8% by mol, a concentration of water of 31.2% by mol, and a concentration of TBA of 18.8% by mol was fed so that the average linear velocity in the first reactor 5 was a value shown in Table 1. The raw material liquid included no recycled TBA, and the Mi/Miii was 2.22.

The reactor inlet temperature of the first reactor 5 was adjusted to 49.2° C., and hydration reaction of isobutylene was performed in conditions of a reactor outlet temperature of 65.0° C., a pressure of 0.860 MPa (G), and an average residence time of 0.642 hours. Next, 27.1% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 58.8° C., a pressure of 0.797 MPa (G), and an average residence time of 1.18 hours. Next, the total amount of the reaction product obtained in the second reactor 7 was fed to the third reactor 9. The reactor inlet temperature of the third reactor 9 was adjusted to 50.0° C., and hydration reaction of isobutylene was performed in conditions of a reactor outlet temperature of 54.0° C., a pressure of 0.800 MPa (G), and an average residence time of 1.18 hours. The Vw in the first reactor 5, and the total conversion rate of isobutylene in all the reactors are shown in Table 1.

The reaction product including TBA, obtained in the third reactor 9, was fed to the TBA separation tower 10, unreacted isobutylene was separated by distillation, and thus an aqueous TBA solution having a concentration of TBA of 64.6% by mol was obtained. The amount of reboiler heating steam required for production of 1000 parts of the aqueous TBA solution is shown in Table 1.

### Example 4

The reaction apparatus, the cation exchange resin and the amount thereof for filling were those as in Example 1.

A raw material liquid having a concentration of isobutylene of 16.0% by mol, a concentration of water of 32.4% by mol, and a concentration of TBA of 22.7% by mol was fed so that the average linear velocity in the first reactor 5 was a value shown in Table 1. The raw material liquid included recycled TBA, and the Mi/Miii was 3.50.

The reactor inlet temperature of the first reactor 5 was adjusted to 54.6° C., and hydration reaction of isobutylene was performed in conditions of a reactor outlet temperature of 64.9° C., a pressure of 0.903 MPa (G), and an average residence time of 0.482 hours. Next, 23.3% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 61.2° C., a pressure of 0.807 MPa (G), and an average residence time of 1.05 hours. Next, the total amount of the reaction product obtained in the second reactor 7 was fed to the third reactor 9. The reactor inlet temperature of the third reactor 9 was adjusted to 53.0° C., and hydration reaction of isobutylene was performed in conditions of a reactor outlet temperature of 58.3° C., a pressure of 0.800 MPa (G), and an average residence time of 1.04 hours. The Vw in the first reactor 5, and the total conversion rate of isobutylene in all the reactors are shown in Table 1.

The reaction product including TBA, obtained in the third reactor 9, was fed to the TBA separation tower 10, unreacted isobutylene was separated by distillation, and thus an aqueous TBA solution having a concentration of TBA of 63.5% by mol was obtained. The amount of reboiler heating steam required for production of 1000 parts of the aqueous TBA solution is shown in Table 1.

### Example 5

The reaction apparatus, the cation exchange resin and the amount thereof for filling were those as in Example 1.

A raw material liquid having a concentration of isobutylene of 14.6% by mol, a concentration of water of 28.0% by mol, and a concentration of TBA of 20.6% by mol was fed so that the average linear velocity in the first reactor 5 was a value shown in Table 1. The raw material liquid included no recycled TBA, and the Mi/Miii was 4.71.

The reactor inlet temperature of the first reactor 5 was adjusted to 58.6° C., and hydration reaction of isobutylene was performed in conditions of a reactor outlet temperature of 65.0° C., a pressure of 1.06 MPa (G), and an average residence time of 0.425 hours. Next, 14.2% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 62.0° C., a pressure of 0.870 MPa (G), and an average residence time of 1.52 hours. Next, the total amount of the reaction product obtained in the second reactor 7 was fed to the third reactor 9. The reactor inlet temperature of the third reactor 9 was adjusted to 52.0° C., and hydration reaction of isobutylene was performed in conditions of a reactor outlet temperature of 56.2° C., a pressure of 0.841 MPa (G), and an average residence time of 1.52 hours. The Vw in the first reactor 5, and the total conversion rate of isobutylene in all the reactors are shown in Table 1.

The reaction product including TBA, obtained in the third reactor 9, was fed to the TBA separation tower 10, unreacted isobutylene was separated by distillation, and thus an aqueous TBA solution having a concentration of 62.6% by mol was obtained. The amount of reboiler heating steam required for production of 1000 parts of the aqueous TBA solution is shown in Table 1.

### Comparative Example 1

The reaction apparatus, the cation exchange resin and the amount thereof for filling were those as in Example 1.

A raw material liquid having a concentration of isobutylene of 12.2% by mol, a concentration of water of 33.8% by mol, and a concentration of TBA of 32.6% by mol was fed so that the average linear velocity in the first reactor 5 was a value shown in Table 1. The raw material liquid included recycled TBA, and the Mi/Miii was 4.92.

The reactor inlet temperature of the first reactor 5 was adjusted to 52.4° C., and hydration reaction of isobutylene was performed in conditions of a reactor outlet temperature of 64.8° C., a pressure of 0.885 MPa (G), and an average residence time of 0.617 hours. Next, 31.0% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 54.1° C., a pressure of 0.812 MPa (G), and an average residence time of 0.999 hours. Next, the total amount of the reaction product obtained in the second reactor 7 was fed to the third reactor 9. The reactor inlet temperature of the third reactor 9 was adjusted to 50.0° C., and hydration reaction of isobutylene was performed in conditions of a reactor outlet temperature of 53.2° C., a pressure of 0.800 MPa (G), and an average residence time of 0.999 hours. The fluid A was in the form of a homogeneous phase in the first reactor 5, and had no isobutylene phase and no aqueous phase. The total conversion rate of isobutylene in all the reactors is shown in Table 1.

The reaction product including TBA, obtained in the third reactor 9, was fed to the TBA separation tower 10, unreacted isobutylene was separated by distillation, and thus an aqueous TBA solution having a concentration of 62.5% by mol was obtained. The amount of reboiler heating steam required for production of 1000 parts of the aqueous TBA solution is shown in Table 1.

### Comparative Example 2

The reaction apparatus, the cation exchange resin and the amount thereof for filling were those as in Example 1.

A raw material liquid having a concentration of isobutylene of 12.0% by mol, a concentration of water of 35.4% by mol, and a concentration of TBA of 31.9% by mol was fed so that the average linear velocity in the first reactor 5 was a value shown in Table 1. The raw material liquid included recycled TBA, and the Mi/Miii was 4.71.

The reactor inlet temperature of the first reactor 5 was adjusted to 52.6° C., and hydration reaction of isobutylene was performed in conditions of a reactor outlet temperature of 65.3° C., a pressure of 0.889 MPa (G), and an average residence time of 0.617 hours. Next, 31.7% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 53.4° C., a pressure of 0.815 MPa (G), and an average residence time of 0.978 hours. Next, the total amount of the reaction product obtained in the second reactor 7 was fed to the third reactor 9. The reactor inlet temperature of the third reactor 9 was adjusted to 50.0° C., and hydration reaction of isobutylene was performed in conditions of a reactor outlet temperature of 53.1° C., a pressure of 0.800 MPa (G), and an average residence time of 0.978 hours. The fluid A was in the form of a homogeneous phase in the first reactor 5, and had no isobutylene phase and no aqueous phase. The total conversion rate of isobutylene in all the reactors is shown in Table 1.

The reaction product including TBA, obtained in the third reactor 9, was fed to the TBA separation tower 10, unreacted isobutylene was separated by distillation, and thus an aqueous TBA solution having a concentration of 61.9% by mol was obtained. The amount of reboiler heating steam required for production of 1000 parts of the aqueous TBA solution is shown in Table 1.

### Comparative Example 3

The reaction apparatus, the cation exchange resin and the amount thereof for filling were those as in Example 1.

A raw material liquid having a concentration of isobutylene of 13.7% by mol, a concentration of water of 34.1% by mol, and a concentration of TBA of 29.4% by mol was fed so that the average linear velocity in the first reactor 5 was a value shown in Table 1. The raw material liquid included recycled TBA, and the Mi/Miii was 3.79.

The reactor inlet temperature of the first reactor 5 was adjusted to 51.1° C., and hydration reaction of isobutylene was performed in conditions of a reactor outlet temperature of 65.1° C., a pressure of 0.902 MPa (G), and an average residence time of 0.610 hours. Next, 35.0% by mass of the reaction product obtained in the first reactor 5 was fed to the second reactor 7. The reactor inlet temperature of the second reactor 7 was adjusted to 50.0° C., and hydration reaction of isobutylene was performed in conditions of a reactor outlet temperature of 54.0° C., a pressure of 0.828 MPa (G), and an average residence time of 0.873 hours. Next, the total amount of the reaction product obtained in the second reactor 7 was fed to the third reactor 9. The reactor inlet temperature of the third reactor 9 was adjusted to 50.0° C., and hydration reaction of isobutylene was performed in conditions of a reactor outlet temperature of 53.3° C., a pressure of 0.800 MPa (G), and an average residence time of 0.873 hours. The Vw in the first reactor 5, and the total conversion rate of isobutylene in all the reactors are shown in Table 1.

The reaction product including TBA, obtained in the third reactor 9, was fed to the TBA separation tower 10, unreacted isobutylene was separated by distillation, and thus an aqueous TBA solution having a concentration of 61.4% by mol was obtained. The amount of reboiler heating steam required for production of 1000 parts of the aqueous TBA solution is shown in Table 1.

TABLE 1

| | | | | Amount of reboiler |
|---|---|---|---|---|
| | Average linear | Volume Vw of | | heating steam |
| | velocity of raw | aqueous phase | Conversion rate | [part(s)/1000 parts |
| | material liquid | [% by volume] | of isobutylene | of aqueous TBA |
| | [m/hour] | in liquid A | [%] | solution] |
| Example 1 | 10.0 | 5.6 | 90.5 | 455 |
| Example 2 | 10.3 | 5.7 | 89.1 | 496 |
| Example 3 | 10.0 | 9.4 | 87.9 | 396 |
| Example 4 | 13.2 | 9.0 | 88.0 | 384 |
| Example 5 | 14.9 | 8.5 | 87.5 | 449 |
| Comparative | 10.3 | 0.0 | 86.5 | 560 |
| Example 1 | | | | |
| Comparative | 10.3 | 0.0 | 87.0 | 545 |
| Example 2 | | | | |
| Comparative | 10.5 | 3.9 | 85.6 | 510 |
| Example 3 | | | | |

As shown in Table 1, each of Examples 1 to 5, in which the average linear velocity and the Vw of the raw material liquid satisfied the above defined conditions, could produce TBA at a higher conversion rate of isobutylene than those in Comparative Examples. It was also indicated that the amount of reboiler heating steam in the TBA separation tower was smaller than those in Comparative Examples and the production cost could be suppressed. Furthermore, the facility cost could be suppressed because of no need for a step of separating carboxylic acid ester of TBA unlike conventional one.

### INDUSTRIAL APPLICABILITY

According to the present invention, tert-butyl alcohol can be obtained at a high conversion rate with suppressed facility cost and production cost.

### DESCRIPTION OF SYMBOLS

1: isobutylene feed port
2: water feed port
3: pump
4: raw material cooler
5: first reactor
6: raw material cooler
7: second reactor
8: raw material cooler
9: third reactor
10: TBA separation tower
11: condenser
12: reboiler
13: unreacted isobutylene discharge port
14: TBA discharge port
15: TBA circulation line

What is claimed is:

1. A method for producing tert-butyl alcohol, comprising
(i) a step of feeding a raw material liquid including isobutylene and water, to a reactor having a cation exchange resin, and
(ii) a step of producing a reaction product including tert-butyl alcohol, by hydration reaction of isobutylene in the reactor, wherein, in step (ii),
the average superficial linear velocity of the raw material liquid, as calculated in the following expression (I), is 5 m/hour or more,
Average linear velocity of raw material liquid (m/hour)=Volume flow rate of raw material liquid (m³/hour)/Cross-sectional area of reactor (m²) (I), and
the volume of an aqueous phase (Vw) in a fluid (fluid A) to the total volume of the fluid A in the reactor is 4 to 12% by volume.

2. The method for producing tert-butyl alcohol according to claim 1, wherein the average superficial linear velocity of the raw material liquid is 5 to 29 m/hour in step (ii).

3. The method for producing tert-butyl alcohol according to claim 1, wherein the average superficial linear velocity of the raw material liquid is 7 to 13 m/hour in step (ii).

4. The method for producing tert-butyl alcohol according to claim 1, wherein the Vw is 5 to 10% by volume in step (ii).

5. The method for producing tert-butyl alcohol according to claim 1, wherein the outlet temperature of the reactor is 75° C. or less in step (ii).

6. The method for producing tert-butyl alcohol according to claim 1, further comprising (iii) a step of separating unreacted isobutylene from the reaction product including tert-butyl alcohol, produced in step (ii), to produce tert-butyl alcohol.

7. The method for producing tert-butyl alcohol according to claim 6, wherein, when the amount of tert-butyl alcohol fed per unit time to the reactor in step (i) is defined as Mi (mol/hour) and the amount of tert-butyl alcohol produced per unit time in step (iii) is defined as Miii (mol/hour), Mi/Miii is 1 to 7.

8. The method for producing tert-butyl alcohol according to claim 7, wherein the Mi/Miii is 1 to 5.
